(19) United States
(12) Patent Application Publication
(10) Pub. No.: US 2025/0279182 A1
(43) Pub. Date: Sep. 4, 2025

(54) Culture For Positive Impact

(71) Applicant: Paresh B. Fozdar, Elmhurst, NY (US)

(72) Inventor: Paresh B. Fozdar, Elmhurst, NY (US)

(21) Appl. No.: 18/510,572

(22) Filed: Nov. 15, 2023

(51) Int. Cl. G16H 20/70

(52) U.S. Cl. CPC G16H 20/70

(57) ABSTRACT

Culture For Positive Impact is applicable in any field for progress and growth, such as sustainability for ecosystem, with realization of moderation with coexistence. Application of Strategy For Positive Impact on need point out wrong, such as human behavior vitiate system and ruin environment for exponential threat in ecosystem: exploration with exploitation such as dominance with deceit, misfire on survival; outplay nature endeavor, such as division of habitat for ease intensify feud and calamity, diminish basic. Iteration of Strategy For Positive Impact on wrong identify right practice for capable, such as analysis and inquiry on threat derive true action for sustainability: guard for moral rights, such as offset of extreme power, claim from abandon and punish violation; aid for viable creative such as consensus on passion for value improvement in honest direction; growth for verifiable advancement such as recovery by reuse from available for believable progress.

### APPROACH

Apply Strategy For Positive Impact (SPI) to analyze and mitigate need, and produce positive impact on society and environment towards sustainability

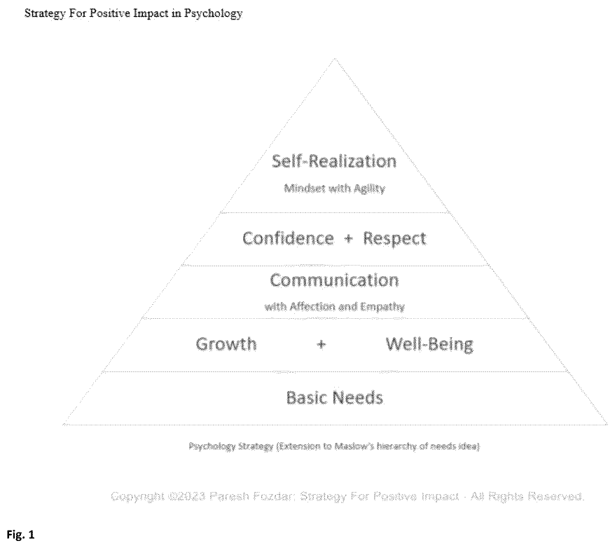

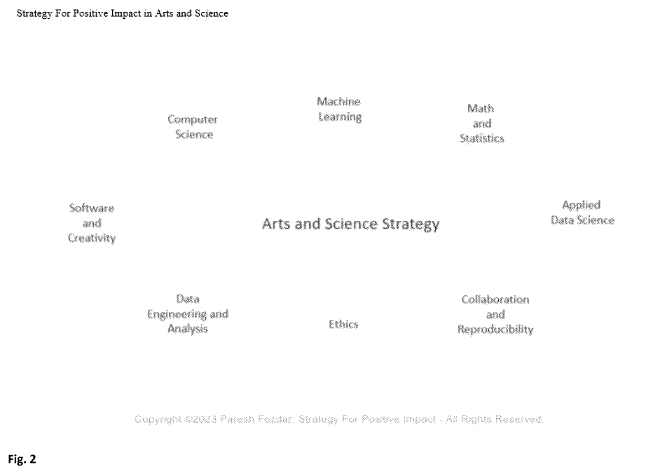

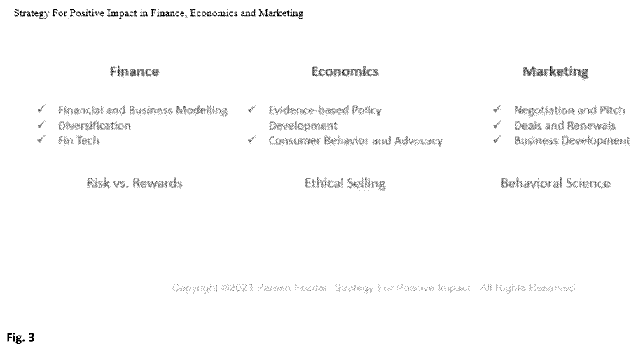

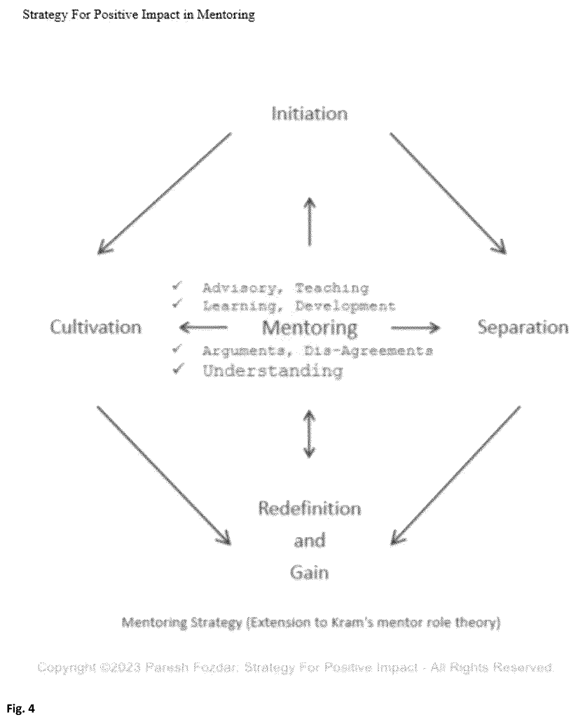

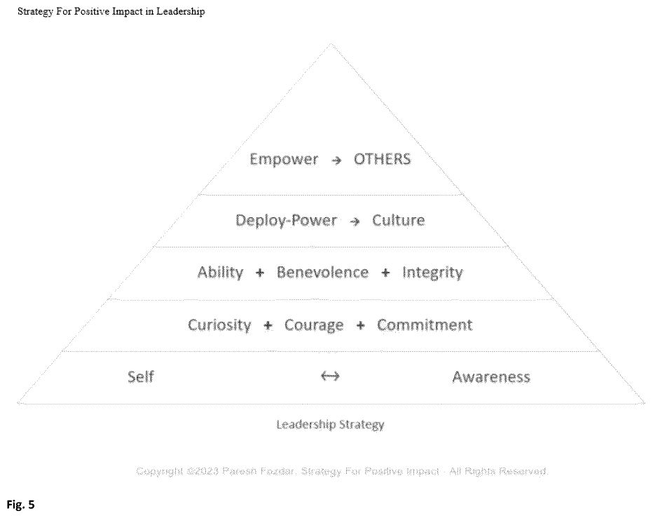

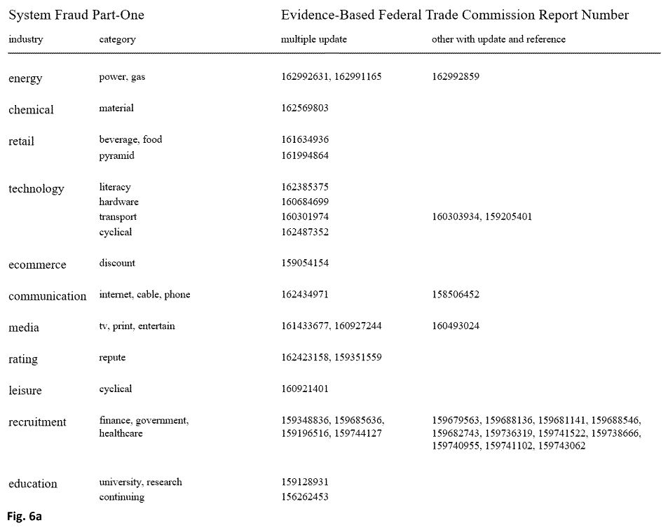

For: SPI
Refer to, FIG. 1 is SPI in Psychology
Refer to, FIG. 2 is SPI in Arts and Science
Refer to, FIG. 3 is SPI in Finance, Economics and Marketing
Refer to, FIG. 4 is SPI in Mentoring
Refer to, FIG. 5 is SPI in Leadership

### COMPONENTS

Need

Practice

Sustain

Impact

### Need

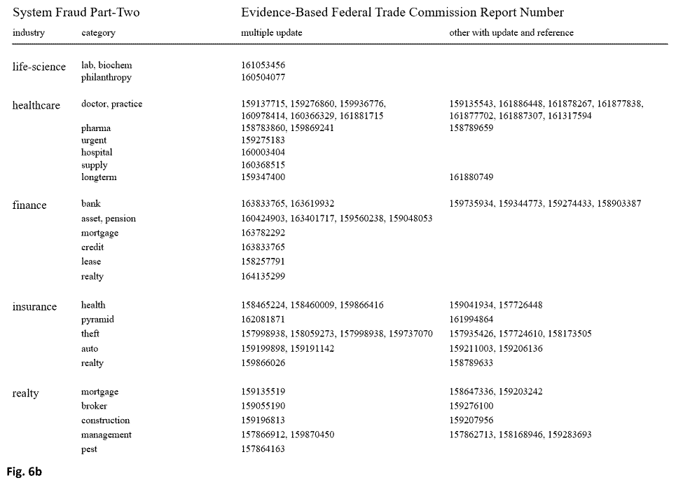

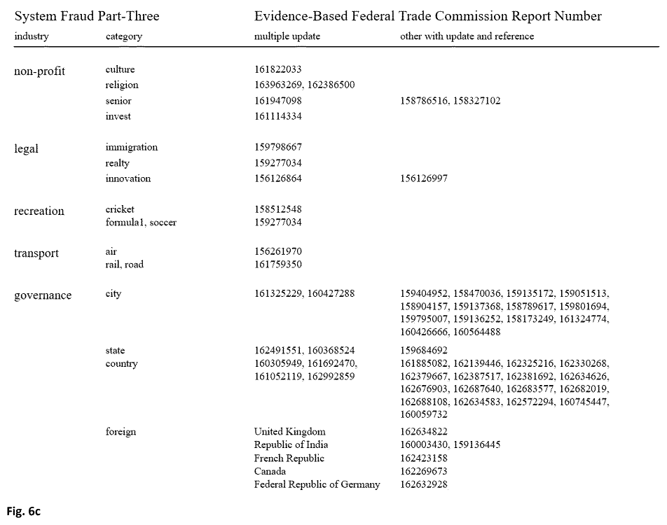

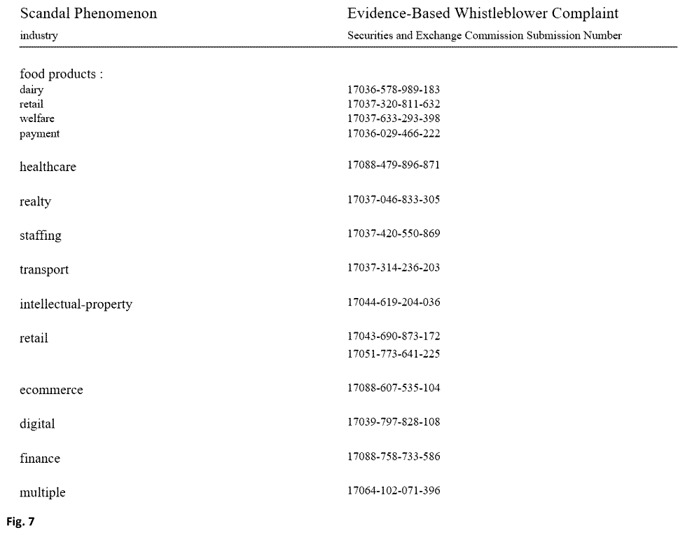

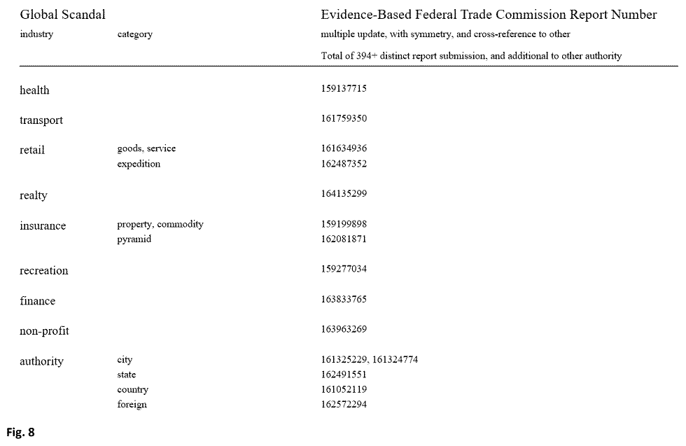

Apply SPI to analyze system
Refer to, FIG. 6a is System Fraud Part-One
Refer to, FIG. 6b is System Fraud Part-Two
Refer to, FIG. 6c is System Fraud Part-Three
Refer to, FIG. 7 is Scandal Phenomenon
Refer to, FIG. 8 is Global Scandal

### Social Harm

Unethical conduct, sufferance and activity to thrive immoral e.g.
greed overpower moral
alter rule for access to security in disaster

### Neglect

1. Contrive ignorance on duty to make competence inapplicable for no functional significance e.g.
delude to evade service with accountability deviant

2. Amass and metastasize compunction for failure to alleviate qualm e.g.
coerce feud or corporate transaction with projection to clear guilty conscience

3. Engulf to accept and align for covert by stratum with requisite to agree and fuel dishonest e.g.
obscure coercion to discourage diversity

### Mistake

1. Nest expertise of prominence to enlarge hierarchy, for disconnect with support to lose purpose e.g.
abet healthcare with insurance wrapper

2. Puncture system for ease to rationalize deliberate goof and disclose as rife e.g.
pick to paralyze by pacemaker or malignant tumor
no bystander response with lost trust

3. Authorize to sponsor problematic concept of influential to establish as norm for ceaseless rise e.g.
overuse of versatile plastic with basis fear
coerce to retain gain congruence since inception

### Enfeeble

1. Deceive intelligence to prop alike with ally collusion for funds e.g.
corporate claim rights to deject novel for exploitation
interpose and negotiate startup idea to lose essence

2. Employ fear with iterative infamy to guard self and enable trade e.g.
directive to secure data as distraction to traffic
derail purpose of security:
   warrant mulct for panic
   sniff detail to poll and price
   bully to raise premium

3. Parade for policy to empower legal and social feud e.g.
advertise system is broken to confess and ploy for transient trust

### Endure

1. Elude veracious with irrelevance and tangent for distrust to propagate paranoia e.g.
dubious goal with relativity for no clarity nor quantification on claim
strenuous pretension to exhibit unavailability
collaborate to blame writ for coordination remiss
scourge professionalism to manifest nonfunctional circus
artificial predict health for illusion of dementia

2. Exploit emotion and forgiveness to realize pity e.g.
dupe, blame and convolute in disguise
bolster bargain and empathy for incompetence
apologize for fitful flaw as defense to continue broken and circle

3. Swindle religion, faith and trust for wealth in guise e.g.
volunteer is conducive for no livelihood
care to quill alliance with trajectory for prospect
oblige to verify recognition to machine

### Eradicate

1. Patrician sell repute to facilitate committee assign for cumulation and concoct power for sovereign e.g.
exploit influence to coerce on other for eminence
directive to aristocrat, for possession of echelon relay order
doyen mule apply recipe to formulate benefit

2. Elect for wile to dictate self-interest e.g.
select to apply policy and breed to coerce
legalize obstruction and automate incapability
delegate with nescience and stopgap for inevitable turmoil to gestate feud
exhibit antagonism with incongruity to claim relinquishment

3. Camouflage to partner, learn, squeeze and acquire with ability to sewer skill and upshot non-lucrative otiose e.g.
collude for accreditation to fraternize and sell disparity
deflect liability by literacy pest
connive for palpable fabrication to compete
fund representation to project belief with perseverance and cycle talent
seize to monopolize and dart oligopoly cartel with promulgation to diversify
intrude to beset and implant trojan as confirmation
manifest despotism by sequel of conglomerate toxin

### Environment Harm

Alter nature to diminish basic

### Disturbance

1. Repurpose habitat with no grasp on lasting effect to biotic and abiotic e.g.
organism breed on air and sour water to migrate for food, plague and aerify to cease

2. Renegade or opportunist ease with exotic import to kill native e.g.
colonist rush to feed labor by coerce growth with faux to dehydrate other

3. Divide for shortcut or nurture feud to threaten self e.g.
perpetuate need for labor to drill passage or raise wall

### Imbalance

1. Starve resource to erupt and reach for quarry e.g.
toll for water
vicious filter for air
invent for lost food
ephemeral pliant for shelter

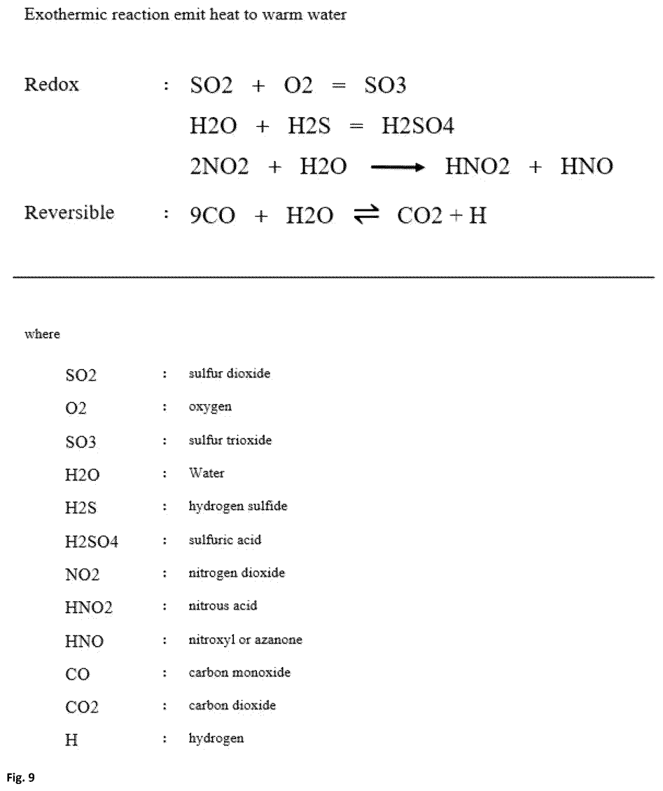

2. Intensify disaster with extreme contrast e.g.
hurricane with shallow gulf refinery to warm water warmer
Refer to, FIG. 9 is an exothermic reaction

3. Facilitate novel to advance, and abet for avarice e.g.
atomic reaction by turbine, in surfeit to exacerbate for covert dross
entomb or bury with harmful to radiate

### Depletion

1. Weaken protection to produce damage e.g.
wither ozone in stratosphere with slow hydrofluorocarbon emission and prior chlorofluorocarbon for excessive ultraviolet rays

2. Hinder process essential to live e.g.
sap marine photosynthesis for oxygen and food by excess use of phytoplankton for polymerization and isomerization

### Analysis

Failure with dependency slip indicate threat to human existence

### Practice

Apply SPI to inquire and learn from failure

### Social Coexistence

Forsake wrong, motivate creative, and guide to believe

### Dispose

1. Abandon broken and restore trust e.g.
renounce irreparable
claim revenue from scam or failure

2. Erode visible clone and inspire diverse e.g.
kill kinship of sameness
eliminate multiple badge for one produce

3. Exercise altruism to exterminate non-profit and stagnate philanthropy e.g.
forbid sect to breed and emerge
fortify explicit to end informal hierarchy to reduce bureaucracy
distrust charity hoax for barter guise

### Cease

1. Psychological projection to deceive e.g.
anchor by availability heuristic of inherent: origin, prominent, name
foreign entity domicile with global satellite offshoot

2. Disregard misconduct for immoral to snowball e.g.
delinquency is testament for evil to replicate and rocket iterative distinct failure, act as witness for criminal transgression or malfeasance

### Concept

1. Reset imagination to tolerate abstract e.g.
realize mindset to internalize novel
enable economic impetus for geographic peculiarity

2. Autonomy with synergy to facilitate and foster new e.g.
propel to ease and pilot
inventor own asset for alliance with no entity possession
concurrence to purpose

3. Purpose at inception with constraint: credible, need, immutable i.e.
assess risk to reward
prohibition on name or repute for: sell, transfer, inherit, claim, merge, acquire
paradigm clarity with vision to decide role and add meaning for function

### Lead

1. Identify passion for skill to achieve task e.g.
listen and understand to inquire and engage
coach and mentor to discover leader

2. Experiment to observe and recognize perspective with empathy e.g.
resolve conflict or barrier for change resistor
train power to license, penalize or revoke
alter with focus to follow purpose

3. Cultivate psychological safety, well-being and inclusion to breathe ethics for trust e.g.
clear goal direction with map to vision
train to deliver value

### Monitor

Autonomy to serve people and resist power of: influence, referent and coercion e.g.
tenure offset with nomination from 7 or higher prime number groups
equal payment with no external fund for elect
distribute to localize for availability with liability
service with purpose to provide for consumer

1. Protect for antitrust e.g.
penalize to discipline and dissolve
sanction to imprison malice, violator or discourteous for purpose misalign
legitimacy to data and conduct maintenance check
reveal to kill bias or deception in: diplomacy, policy, transaction

2. Govern for growth e.g.
analyze capability and vulnerability at intra, inter and foreign to negotiate
direct allocation by region to correspond issuance with productivity
align for agile decision-making ability to change

3. Surveil for neutrality e.g.
intelligence insight to end camouflage of harm, threat or deception
force for defense and offense to secure for unity and flag

### Environment Moderation

Recover basic for verifiable growth e.g.
restore by discharge of essential
retain to repurpose vital
reduce distress

### Water

Embrace balance to approach salvage

1. Reprocess

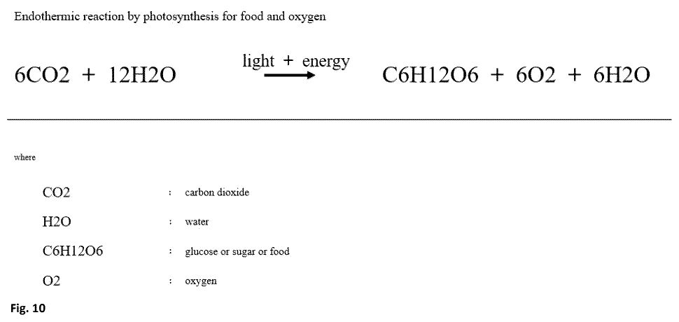

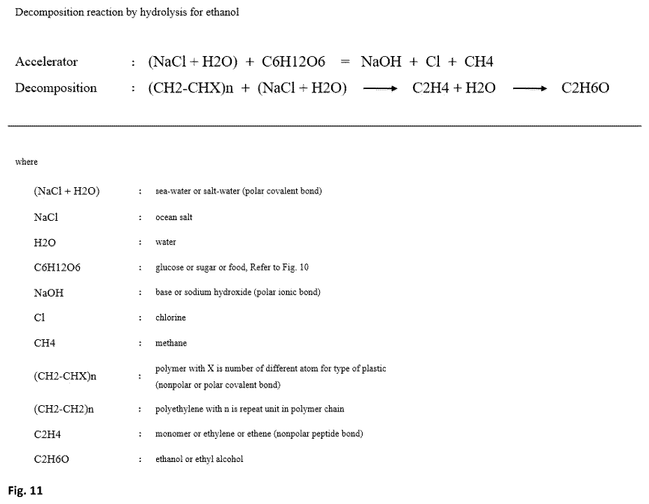

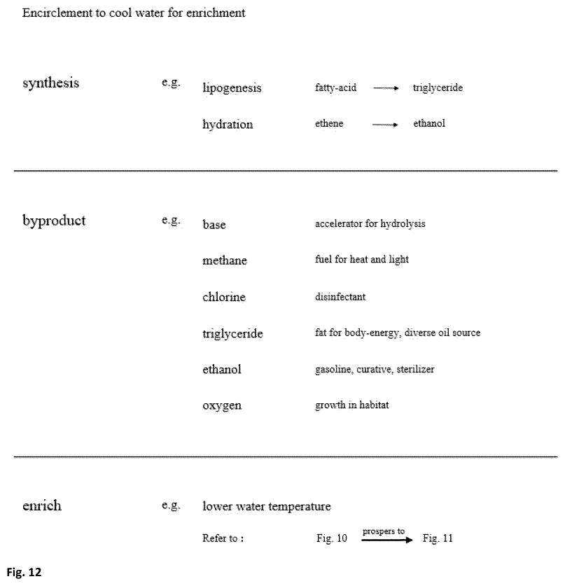

Weaken hurricane by reuse of heat at gulf to stimulate endothermic reaction e.g.
light and energy by chemiluminescence for photosynthesis
spinoff for crucial: food and oxygen
Refer to, FIG. 10 is an endothermic reaction

2. Reclaim

Outwit flood by speedy fragmentation of onerous waste to benign biodegrade for reuse e.g.
ocean water with food provide accelerator for decomposition by hydrolysis
break complex plastic to simpler structure
offshoot for multiuse: ethanol
Refer to, FIG. 11 is decomposition reaction

3. Abate

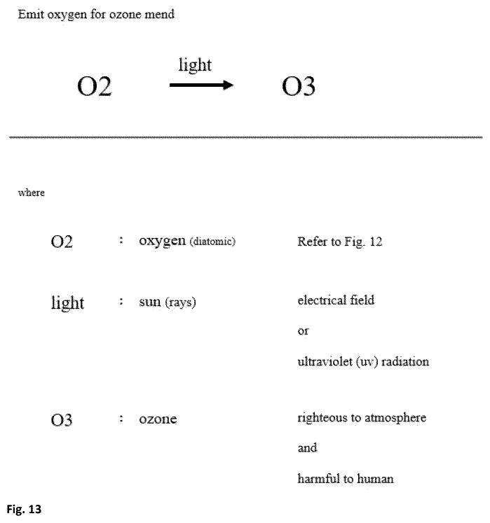

Diminish disaster and calamity by iterative reprocess to reclaim and affect ocean e.g.
redirect heat from water to reduce temperature
emit oxygen to further cool down
absorb chemical product to decay by synthesis
derivative for beneficial: byproduct
encircle action to quench and besiege for prosperous: habitat enrichment
Refer to, FIG. 12 is an encirclement

### Air

Remit assent for equilibrium e.g.
ransom of diatomic oxygen hit light ray from sun to self-heal ozone
automatic mend to elude harmful
Refer to, FIG. 13 is ozone mend

### Shelter

1. Growth spread with season e.g.
migrate to search food
hunt to predate on wild
build coral reef to atoll, isle to island

2. Rehabilitate to evolve in favorable e.g.
subdue incorrect, unfit and non-native invasion
cease to remodel real with natural in abundance

### Inquiry

Exploration of failure and harm to discover interdependent moderation with coexistence dictate basis for human existence

### Sustain

Acknowledge moderation with conscious for coexistence to change for progress e.g.
live and let live with air, water and food in habitat

For word: practice
Refer to Component: Practice

### Accept

Adapt to adjust for variance with no correlation to glacial epoch or past eon

1. Present differ from past to speculate future with no witness nor model nor basis e.g.
deactivate change of extraneous circumstance to mint with era

2. Vital pedagogy to develop character for perception and passion to flourish e.g.
school with introspection for explicable science to assess interest and curiosity for growth
embrace to attempt by practice before ambitious propagation to compete and amplify

3. Large predate on small to exist and grow, defunct with oblivion or greed e.g.
specie extinction, or merger and acquisition
ignorance towards delegate with act as convoy empower:
   disloyal to ripple as wave
   trojan to tide and flood
   region to crumble by hypocrisy

### Admit

Respect power of nature to advance life via metamorphosis to:

1. Attain eternal power to virtualize intelligence with viral mutation, to brace as prey or self-immolate e.g.
destroy nature with specie extinction to continue former

2. Mutualism division by extremism and fanaticism strike imbalance for commensalism lead to parasitism e.g.
community divide by coerce bias group produce unrest in moral reef

3. Co-exist and survive to convey next e.g.
permian to dinosaur to mammalia to successor

### Aware

Mindful discard, as inevitable rebound to imbue for genesis e.g.

1. Defiant
innovation as transformation of past failure to hound
plastic in ocean as food guise to marine ensue as catch to feed human for woe

2. Incorrect
utilize inorganic to dim or swell
alter native to inherit foreign charm

3. Mar
underwrite risk by stale correlate to harbor for uncertain stability
wager resilience by regulation with fictional target
gene implant for mutation to lose indigenous

### Annihilate

Obliterate: violation, fraud, theft, defamation and antitrust e.g.

1. Spot to assess and destroy
ambiguity
breach of reputational-trust

2. Discern implicit or explicit to discipline
price-fixing
unfairness

3. Cease and desist to extirpate
manipulation
abuse
misrepresentation
restraint
harassment

### Advance

Dream to realize and model with ethical efficiency for moral effectiveness, by practice e.g.

1. Diversity and equity with legitimate pressure
disintegrate monopoly and conglomerate
detach expert power to collaborate for alliance

2. Reinforce society
entertain by funnel of aspiration
invest or save to protect for value and produce opportunity

3. Actualize creativity
anchor conflict for transparency
evade exhaustion to perform
abide with determination for purpose

### Change

Commitment to understand impact of SPI and practice for transition derive sustainability in ecosystem

### Impact

Iterative application of SPI to need with analysis and inquiry to identify and realize positive contour institute Culture For Positive Impact

1. Culture For Positive Impact is applicable in any field for progress and growth, such as sustainability for ecosystem, with realization of moderation with coexistence.

2. Application of Strategy For Positive Impact on need point out wrong, such as human behavior vitiate system and ruin environment for exponential threat in ecosystem:
a. Exploration with exploitation such as dominance with deceit, misfire on survival;
b. Outplay nature endeavor, such as division of habitat for ease intensify feud and calamity, diminish basic.

3. Iteration of Strategy For Positive Impact on wrong identify right practice for capable, such as analysis and inquiry on threat derive true action for sustainability:
a. Guard for moral rights, such as offset of extreme power, claim from abandon and punish violation;
b. Aid for viable creative such as consensus on passion for value improvement in honest direction;
c. Growth for verifiable advancement such as recovery by reuse from available for believable progress.
